(19) United States
(12) Patent Application Publication
(10) Pub. No.: US 2005/0129645 A1
(43) Pub. Date: Jun. 16, 2005

(54) Hair shaping composition comprising at least one non-hydroxide imine

(75) Inventors: Gerard Malle, Villiers S/Morin (FR); Damien Lavergne, Paris (FR); Michel Philippe, Wissous (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL

(21) Appl. No.: 10/989,620

(22) Filed: Nov. 17, 2004

Related U.S. Application Data
(60) Provisional application No. 60/562,539, filed on Apr. 16, 2004.

(30) Foreign Application Priority Data
Nov. 18, 2003 (FR) 03 50856

(51) Int. Cl. A61K 7/11; A61K 7/06; A61K 7/09

(52) U.S. Cl. 424/70.2; 424/70.14; 424/70.12; 424/70.13

(57) ABSTRACT

The disclosure relates to ready-to-use cosmetic compositions for permanently shaping keratin fibers comprising, as a permanent hair-shaping active agent, an imine not belonging to the hydroxide family. The disclosure also provides kits comprising compartments to be placed in contact to form the ready-to-use compositions, and processes using these compositions.

This application claims benefit of U.S. Provisional Application No. 60/562,539, filed Apr. 16, 2004, hereby incorporated by reference.

The present disclosure relates to ready-to-use cosmetic compositions for permanently shaping keratin fibers, comprising, as a permanent hair-shaping active agent, an imine not belonging to the hydroxide family. The present disclosure also relates to kits comprising compartments to be placed in contact to form the ready-to-use compositions, and also to processes using the compositions.

The term “imine not belonging to the hydroxide family,” as used herein, encompasses any organic compound:
having at least one carbon atom double-bonded to a nitrogen atom and single-bonded to another nitrogen atom and single-bonded to another carbon atom or to a third nitrogen atom;
containing no hydroxide groups in its chemical formula; and
capable of accepting a proton.
   The term also includes organic and mineral salts of such compounds. These “imines not belonging to the hydroxide family” include guanidines and amidines.

The term “keratin fibers,” as used herein, encompasses keratin materials, such as fibers of human or animal origin such as head hair, other body hairs, eyelashes, wool, angora, cashmere, fur and the like. Througout the specification, reference is made to head hair, although the disclosure is not limited to particular keratin fibers.

The term “permanent shaping,” as used herein, includes the curling, permanent-waving or setting of keratin fibers, for example, Caucasian, Asiatic or North African hair.

The expression “between A and B,” as used herein, means ranging from A to B, the limits A and B being included.

Two techniques may be used to permanently reshape the hair. They are based on breaking the cystine disulfide bonds present in keratin. It will be understood that these techniques can be successfully employed, even if not all of the cystine disulfide bonds are broken.

The first technique comprises opening the disulfide bonds using a composition comprising a reducing agent, optionally rinsing the hair, placing the hair under tension with curlers or the like, or shaping or smoothing out by other means, and reconstituting the disulfide bonds by applying an oxidizing composition (also known as a fixing composition) to the hair, so as to give the head of hair the desired shape. This technique makes it possible either to permanently shape the hair or to relax it, decurl it or smooth it out.

The second technique comprises performing a lanthionization reaction, using a composition comprising a base belonging to the hydroxide family. This leads to replacement of disulfide bonds (—CH₂—S—S—CH₂—) with lanthionine bonds (—CH₂—S—CH₂—). The lanthionization reaction involves two consecutive chemical reactions:
The first reaction comprises a beta-elimination on the cystine brought about by a hydroxide ion, leading to the breaking of the bond and the formation of dehydroalanine. 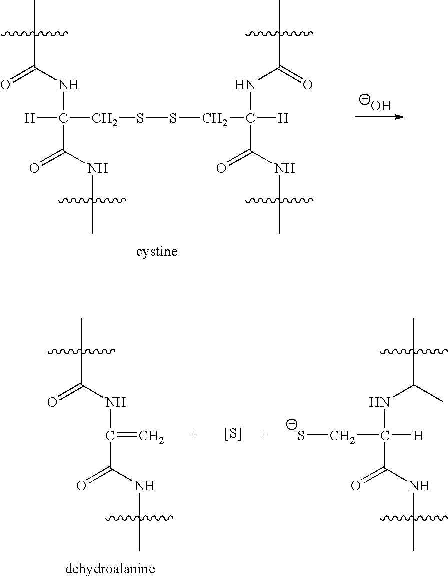
The second reaction comprises a reaction of the dehydroalanine with a thiol group. The double bond of the dehydroalanine formed is a reactive double bond that can react with the thiol group of the cysteine residue that has been released to form a new bond, referred to as a lanthionine bridge or bond or residue.

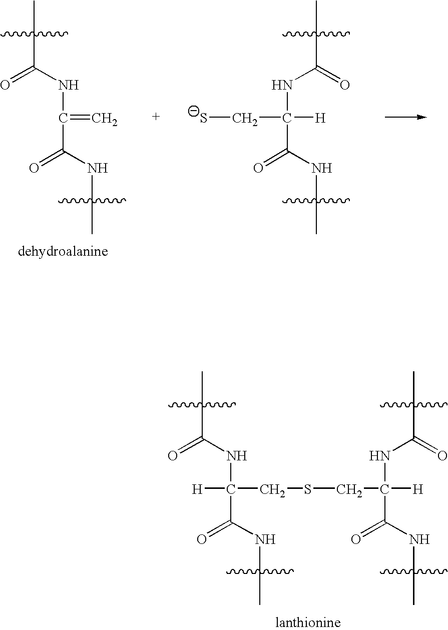

Compared to the first technique, which uses a reducing agent, the lanthionization technique does not require a fixing step, because the formation of the lanthionine bridges is irreversible. It therefore takes place in a single step and makes it possible either to make the hair wavy, relax it, decurl it, or straighten it out. However, it is mainly used to relax naturally frizzy or curly hair.

In the first technique, the reducing compositions generally used for the first step of a permanent-waving or relaxing operation contain thiols, sulfites or bisulfites as reducing agents. These agents are generally used in essentially aqueous medium at concentrations ranging from 0.5M to 1 M to obtain good opening of the disulfide bonds. Thiols that may be used include, but are not limited to, thioglycolic acid, cysteamine, glyceryl monothioglycolate, thiolactic acid and cysteine. Thioglycolic acid, for instance, is efficient at reducing keratin disulfide bonds at alkaline pH, such as in the form of ammonium thioglycolate, and is the product most commonly used in permanent-waving (hair waving). However, it has been found that thioglycolic acid must be used in a sufficiently basic medium (in practice at a pH ranging from 8.5 to 9.5) if it is desired to obtain curling of sufficient intensity. In addition to the drawback of releasing an unpleasant odor requiring the use of fragrances to mask the odor, the use of thiols at alkaline pH also may lead to degradation of fibers and to loss of any artificial colorations.

Sulfites and bisulfites are mainly used for relaxing. They have drawbacks similar to those of thiols, and may have lower efficacy.

Thiols, sulfites, and bisulfites also may have the drawback of having poor stability in aqueous solution.

In general, the durability of the reshaping effects obtained with thiols and sulfites by reduction of disulfides followed by fixing is considered inferior to the effects obtained via the lanthionization technique.

The compositions generally used to perform the lanthionization technique contain as a base a hydroxide such as sodium hydroxide, guanidinium hydroxide, and lithium hydroxide. These lanthionization active agents, which make it possible to open disulfide bonds via a beta-elimination reaction, are generally used in a water-in-oil emulsion at concentrations ranging from 0.4M to 0.6M, by leaving them to act generally for 10 to 15 minutes at room temperature. Sodium hydroxide is the agent most commonly used. Guanidinium hydroxide is also commonly used and may be preferred in many such compositions. Sodium hydroxide and guanidinium hydroxide are the two most common agents used for relaxing or decurling naturally frizzy or curly hair. They may have several advantages over ammonium thioglycolate and sulfites, including the absence of an unpleasant odor, the fact that only one implementation step is required resulting in shorter treatment time, and much greater durability and efficacy of the reshaping of the hair.

However, these hydroxides have a major drawback of being caustic. This causticity affects the scalp by causing irritation that may be severe. This may be partially overcome by the prior application to the scalp of a greasy protective cream often referred to as “base” or “base cream”, the word “base” used here not having the meaning of a basic agent in the chemical sense. When the protective cream is combined with the hydroxide in a single composition, this is generally referred to as a “no-base” composition. This “no-base” technology is more convenient.

The causticity of hydroxides also affects the state of the hair by giving it a coarse feel and making it much more fragile. The fragility may make the hair fray, break or even dissolve if the treatment is prolonged. In some cases, hydroxides may also cause decoloration of the natural color of the hair.

Formulations containing sodium hydroxide are generally referred to as “lye relaxers” and those not containing it are generally referred to as “no-lye relaxers”.

The “no-lye” relaxing formulations commonly use guanidinium hydroxide. Since guanidinium hydroxide is unstable, it is generated extemporaneously by mixing guanidine carbonate and a source of very sparingly soluble hydroxide such as calcium hydroxide. The reaction between these two compounds leads to the formation of guanidinium hydroxide and calcium carbonate, which precipitates in the composition. The presence of this precipitate makes the final rinsing of the hair much more difficult and leaves mineral particles on the hair and scalp that give the hair a coarse feel and an unaesthetic appearance resembling dandruff. The recent preference for guanidinium hydroxide (“no-lye”) over sodium hydroxide (“lye”) relaxing formulations appears to arise from better relaxing efficacy and better skin tolerance. However, these technologies using bases of the hydroxide family are still very aggressive treatments to the hair and scalp, and require very strict control of the application time to avoid excessive irritation and impairment of the hair that may go as far as breaking. The aggressiveness arising from the causticity of hydroxides is one reason that these compositions for the lanthionization of the hair are not to be used for permanent-waving (hair waving), but instead are generally reserved for relaxing (hair straightening or hair relaxing).

Furthermore, hydroxides are known to be good agents for hydrolyzing amide functions (see, for example, March's Advanced Organic Chemistry, 5th Edition, Wiley Interscience, New York, “Hydrolysis of Amides,” pages 474 et seq), which thus lead to the breaking of the peptide bonds by direct nucleophilic attack. The impairments observed in the case of hair and keratin materials in the broad sense may largely be due to partial hydrolysis of the amide bonds of keratin.

In light of the above problems, there remains a real need for shaping compositions that are less aggressive to the hair and the skin, including the scalp.

Various studies have been performed to attempt to simultaneously overcome the drawbacks of reducing agents (used in the first technique) and/or of hydroxides (used in the second technique).

Many reducing agents have been proposed to replace thioglycolic acid, but thioglycolic acid in its ammonium thioglycolate form remains both the reference compound and the compound most widely used in cosmetic formulations, for permanently shaping the hair.

However, the use of reducing agents leads to inadequate durability for permanent shaping, meaning that the treatment must be repeated frequently and that there are inevitably instances of damage as treatment is repeated. In addition, the causticity of hydroxides limits their use strictly to the field of hair relaxing. No study has found a base that would be both effective as a permanent hair-shaping active agent and less aggressive than soda lye or hydroxides in general.

After extensive studies, it has now been discovered, entirely surprisingly and unexpectedly, that the first step of the lanthionization process can be performed with imines not belonging to the hydroxide family. Excellent results in terms of intensity of permanent shaping, cosmetic and mechanical properties of the hair are thus obtained, with a durability greater than that obtainable with ammonium thioglycolate, the reference product for permanent shaping.

Thus, in one aspect, the disclosure provides cosmetic compositions comprising, in a cosmetically acceptable medium, at least one imine not belonging to the hydroxide family, the medium and the at least one imine being chosen such that the at least one imine is reactive with the cystines of keratin fibers, via a beta-elimination reaction, to produce dehydroalanine and lead to the formation of lanthionine, to produce with the keratin fibers curls ranging from 0.2 cm to 3 cm in diameter in less than 60 minutes.

The permanent shaping time may be less than 40 minutes, such as less than 30 minutes.

Imines that may be used include guanidines and amidines, such as, but not limited to:
1) Guanidine-type imines,
   a) such as:
N,N-dimethylguanidine
N,N′,N″-trimethylguanidine
N-methylguanidine
N,N-diethylguanidine
N-ethylguanidine
N,N-dimethylimidodicarbonimidic diamide
N-(tert-butyl)-N″-methylguanidine
N-(4-aminobutyl)guanidine
1,3,4,6,7,8-hexahydro-2H-pyrimido[1,2-a]pyrimidine
N,N″-diisopropylguanidine
1-methyl-1,3,4,6,7,8-hexahydro-2H-pyrimido[1,2-a]pyrimidine
N″-(tert-butyl)-N,N,N′,N′-tetramethylguanidine
N,N,N′,N′-tetraethylguanidine
N,N″-di-tert-butylguanidine
N″-[amino(imino)methyl]-N-phenylguanidine
N,N′,N″-triisopropylguanidine
pyrrolidine-1-carboximidamide
N-phenylguanidine
N-(tert-butyl)-N′,N″-dimethylguanidine
N-(4-{[amino(imino)methyl]amino}butyl)guanidine
N″-(tert-butyl)-N-methyl-N′-phenylguanidine
N, N′,N″-tricyclohexylguanidine
N-{3-[(4-{[amino(imino)methyl]amino}butyl)amino]propyl}guanidine
N-(2-hydroxyethyl)-N-methylguanidine
N-(10-{[amino(imino)methyl]amino}decyl)guanidine
hydrazinecarboximidamide
N-hydroxyguanidine
hydrazinecarboximidohydrazide
{[amino(imino)methyl]amino}acetic acid
N-[amino(imino)methyl]thiourea
N-(1,3-dimethyl-1,3-dihydro-2H-imidazol-2-ylidene)-N-methylamine
triazane-2-carboximidamide
morpholine-4-carboximidamide
N-nitroimidazolidin-2-imine
[[amino(imino)methyl](methyl)amino]acetic acid
3-{[amino(imino)methyl]amino}propanoic acid
(2S)-2-{[amino(imino)methyl]amino}propanoic acid
5-methoxy-1,2,4-thiadiazol-3-amine
3-amino-1,2,4-thiadiazole-5-thiol
N-(3-chloropropyl)guanidine
N″-cyclopentylidenecarbonohydrazonic diamide
(2-iminoimidazolidin-1-yl)acetic acid
morpholine-4-carboximidohydrazide
4-{[amino(imino)methyl]amino}butanoic acid
3-[[amino(imino)methyl](methyl)amino]propanoic acid
3-{[imino(methylamino)methyl]amino}propanoic acid
N-hydroxymorpholine-4-carboximidamide
{2-[amino(imino)methyl]hydrazino}(oxo)acetic acid
1H-pyrazole-1-carboximidamide
5-(methylsulfanyl)-1,2,4-thiadiazol-3-amine
1H-1,2,4-triazole-1-carboximidamide
N-(2-sulfanylethyl)guanidine
N-(4-methyl-1,3-thiazol-2-yl)guanidine
2-amino-6-sulfanylpyrimidine-4(3H)-thione
N-(4-oxo-1,3-thiazolidin-2-yl)guanidine
6-amino-4-imino-4H-1,3,5-thiadiazine-2-thiol
2,5-dimethylpyrazolo[1,5-c]pyrimidin-7-amine
2H-1,2,4,6-thiatriazine-3,5-diamine 1,1-dioxide
(2E)-2-(pyrid-2-ylmethylene)hydrazinecarboximidamide
(2R)-2-{[amino(imino)methyl]amino}-3-sulfanylpropanoic acid
triazane-2-carboximidohydrazide
2-{[amino(imino)methyl]amino}ethanesulfonic acid
2-amino-1,7-dihydro-6H-purine-6-thione
N″-(4-chlorophenyl)guanidine
N-[imino(morpholin-4-yl)methyl]guanidine
6-{[amino(imino)methyl]amino}hexanoic acid
(2S)-2-{[amino(imino)methyl]amino}succinic acid
(2S)-5-{[amino(imino)methyl]amino}-2-hydroxypentanoic acid
5-(propylsulfanyl)-1,2,4-thiadiazol-3-amine
(2S)-2-amino-4-({[amino(imino)methyl]amino}oxy)butanoic acid
(2S)-2-amino-4-{[(hydroxyamino)(imino)methyl]amino}butanoic acid
4-{[amino(imino)methyl]amino}benzoic acid
6-ethoxy-3,9-dihydro-2H-purin-2-imine
N-(3-nitrophenyl)guanidine
(2S)-2-amino-3-{[amino(imino)methyl]amino}propanoic acid
N-(2-amino-6-chloropyrimidin-4-yl)guanidine
N-(1,3-dimethyl-1,3-dihydro-2H-imidazol-2-ylidene)-N-phenylamine
(2S)-2-amino-6-{[amino(imino)methyl]amino}hexanoic acid
2-{[amino(imino)methyl]amino}pentanedioic acid
(2S)-2-{[amino(imino)methyl]amino}pentanedioic acid
[[amino(imino)methyl](2,3-dihydroxypropyl)amino]acetic acid
4-hydroxy-3,4-dihydroisoquinoline-2(1H)-carboximidamide
N-(2-methylphenyl)imidodicarbonimidic diamide
N-(tert-butyl)-N″-phenylguanidine
N5-phenyl-1,2,4-thiadiazole-3,5-diamine
(2S)-5-{[amino(imino)methyl]amino}-2-chloropentanoic acid
N-butylimidodicarbonimidic diamide
N-(2-methyl-5-nitrophenyl)guanidine
2-[(6-amino-1H-purin-8-yl)amino]ethanol
N-[amino(imino)methyl]-N′-phenylthiourea
2-hydrazino-1,4,5,6-tetrahydropyrimidine
N′-nitro-2-phenylhydrazinecarboximidamide
N-(4,6-dimethylpyrimidin-2-yl)-N″-methoxyguanidine
6-(ethylsulfanyl)-1H-purin-2-amine
(2S)-2-amino-4-{[amino(imino)methyl]amino}butanoic acid
(2E)-2-(4-chlorobenzylidene)hydrazinecarboximidamide
2-[amino(imino)methyl]-N-ethylhydrazinecarbothioamide
4-({[amino(imino)methyl]amino}methyl)cyclohexanecarboxylic acid
N-(1-naphthylmethyl)guanidine
N,N″-dineopentylguanidine
3,5-dimethyl-1H-pyrazole-1-carboximidamide
N-(4-methylquinazolin-2-yl)guanidine
N-(4-hydroxyquinazolin-2-yl)guanidine
N-(1,3-dimethyltetrahydropyrimidin-2(1H)-ylidene)-N-phenylamine
N-(4-chlorophenyl)guanidine
(2E)-2-(1,3-benzodioxol-5-ylmethylene)hydrazinecarboximidamide
N-(4,6-dimethylpyrimidin-2-yl)-N″-ethoxyguanidine
N,N′-diphenylguanidine
N-phenylimidodicarbonimidic diamide
4-amino-N-[amino(imino)methyl]benzenesulfonamide
ethyl 2-{[amino(imino)methyl]amino}-1,3-thiazole-4-carboxylate
N-(4,6-dimethylquinazolin-2-yl)guanidine
N-(4,7-dimethylquinazolin-2-yl)guanidine
N-(4,8-dimethylquinazolin-2-yl)guanidine
6,6-dimethyl-N-2-phenyl-1,6-dihydro-1,3,5-triazine-2,4-diamine
N-(tert-butyl)-N-(1,3-dimethyl-1,3-dihydro-2H-benzimidazol-2-ylidene)amine
N-(3,4-dichlorobenzyl)guanidine
N″-(4-phenyl-1,3-thiazol-2-yl)guanidine
(3-ethyl-2-imino-2,3-dihydro-1H-benzimidazol-1-yl)acetic acid
1H-perimidin-2-amine
N-(tert-butyl)-N″-methoxy-N′-phenylguanidine
5-(benzylsulfanyl)-1,2,4-thiadiazol-3-amine
N-[2-(dimethylamino)-5-nitropyrimidin-4-yl]guanidine
(1Z,2R)-2-amino-5-{[amino(imino)methyl]amino}-N-hydroxypentanimidic acid
N″-(4-chlorophenyl)-N,N′-diethylguanidine
(2E)-2-(pyrid-4-ylmethylene)hydrazinecarboximidamide
N-[2-(3-chlorophenoxy)ethanimidoyl]guanidine
N-(heptafluoropropyl)guanidine
[(carboxymethyl)(methyl)amino](imino)methylamidophosphate
1,3-dimethyl-2-(methylamino)-4,5-dihydro-1H-imidazole
N-(4-methylphenyl)imidodicarbonimidic diamide
N′-[(2Z)-1-methylpyrimidin-2(1H)-ylidene]benzohydrazide
4-(aminomethyl)piperidine-1-carboximidamide
N-(4,6-dimethylquinazolin-2-yl)-N″-methylguanidine
N-(4,7-dimethylquinazolin-2-yl)-N″-methylguanidine
5-{[amino(imino)methyl]amino}-2-chloropentanoic acid
N-(2,6-dichlorophenyl)-4,5-dihydro-1H-imidazol-2-amine
(2E)-2-(2,6-dichlorobenzylidene)hydrazinecarboximidamide
(2E)-2-(2,4-dichlorobenzylidene)hydrazinecarboximidamide
N-(6-methoxy-4-methylquinazolin-2-yl)guanidine
N-(7-methoxy-4-methylquinazolin-2-yl)guanidine
N-(8-methoxy-4-methylquinazolin-2-yl)guanidine
6,6-dimethyl-N-2-(4-methylphenyl)-1,6-dihydro-1,3,5-triazine-2,4-diamine
N-(4-fluorophenyl)imidodicarbonimidic diamide
N-(3-fluorophenyl)imidodicarbonimidic diamide
N-(2-fluorophenyl)imidodicarbonimidic diamide
N″-(3,4-dichlorophenyl)-N,N′-dimethylguanidine
N-[2-(5-methoxy-1H-indol-3-yl)ethyl]guanidine
N′-(1,3-dimethylimidazolidin-2-ylidene)benzohydrazide
N-(5-bromo-4-methyl-1,3-thiazol-2-yl)guanidine
2-[(2-{[amino(imino)methyl]amino}ethyl)sulfanyl]succinic acid
N-(4,6-dimethylpyrimidin-2-yl)-N″-isopentylguanidine
N-[(5-nitro-2-furyl)methyl]hydrazinecarboximidamide
2,6-dimethylpiperidine-1-carboximidamide
N-(1,3-dimethyl-1,3-dihydro-2H-benzimidazol-2-ylidene)-N-phenylamine
3-[(2-{[amino(imino)methyl]amino}ethyl)sulfanyl]-2-(sulfanylmethyl)propanoic acid
(E)-N′,2-diphenyldiazenecarbohydrazonamide
N-(1,3-dimethyl-1,3-dihydro-2H-benzimidazol-2-ylidene)methanesulfonamide
N,N′-bis(2-methylphenyl)guanidine
sodium 4-[(2-{[amino(imino)methyl]amino}ethyl)sulfanyl]4-oxobutanoate
1,3-dimethyl-2-(methylamino)-3,4,5,6-tetrahydropyrimidine
4-chloro-N4,5-dihydro-1H-imidazol-2-yl-6-methoxy-2-methylpyrimidin-5-amine
N-(2-phenylethyl)imidodicarbonimidic diamide
N-(2,3-dimethylphenyl)imidodicarbonimidic diamide
N-(4-hydroxy-6-methylpyrimidin-2-yl)-N′-phenylguanidine
N9-phenyl-6,8,10-triazaspiro[4.5]deca-6,8-diene-7,9-diamine
5-[(3aS,4S,6aR)-2-iminohexahydro-1H-thieno[3,4-d]imidazol-4-yl]pentanoic acid
N-(3-methoxyphenyl)imidodicarbonimidic diamide
N-(2-methoxyphenyl)imidodicarbonimidic diamide
N-(4-methoxyphenyl)imidodicarbonimidic diamide (2E)-2-[1-(2,4-dichlorophenyl)ethylidene]hydrazinecarboximidamide
N-(8-ethoxy-4-methylquinazolin-2-yl)guanidine
N-(1,2-di-tert-butyldiiaziridin-3-ylidene)-N-phenyl amine
(2S)-3-{[amino(imino)methyl]amino}-2-[(tert-butoxycarbonyl)amino]propanoic acid
N2-(2-methoxyphenyl)-6,6-dimethyl-1,6-dihydro-1,3,5-triazine-2,4-diamine
N,N′-di-tert-butyl-N″-phenylguanidine
N-(2-chlorophenyl)imidodicarbonimidic diamide
N-(3-chlorophenyl)imidodicarbonimidic diamide
N-(4-chlorophenyl)imidodicarbonimidic diamide
N-1-naphthylguanidine
N″-[4-(4-methoxyphenyl)-1,3-thiazol-2-yl]guanidine
N-{imino[2-(1-methylethylidene)hydrazino]methyl}-N′-phenylthiourea
N-(2,4-difluorophenyl)imidodicarbonimidic diamide
N-(2,6-difluorophenyl)imidodicarbonimidic diamide
N-(3,5-difluorophenyl)imidodicarbonimidic diamide
N-(2,5-difluorophenyl)imidodicarbonimidic diamide
N-(3,4-difluorophenyl)imidodicarbonimidic diamide
(2E)-2-[4-(dimethylamino)-3-nitrobenzylidene]hydrazinecarboximidamide
N2-(2-chlorophenyl)-6,6-dimethyl-1,6-dihydro-1,3,5-triazine-2,4-diamine
N2-(4-chlorophenyl)-6,6-dimethyl-1,6-dihydro-1,3,5-triazine-2,4-diamine
N-[4-(4-chlorophenyl)-1,3-thiazol-2-yl]guanidine
N″-[4-(4-chlorophenyl)-1,3-thiazol-2-yl]guanidine
6,6-dimethyl-1-phenyl-1,6-dihydro-1,3,5-triazine-2,4-diamine
(1E)-1-(2-methylphenyl)ethanone [(2Z)-4,6-dimethylpyrimidin-2(1H)-ylidene]hydrazone
(1Z)-1-(2-methylphenyl)ethanone [(2E)-4,6-dimethylpyrimidin-2(1H)-ylidene]hydrazone
disodium [(carboxymethyl)(methyl)amino](imino)methyl amidophosphate
N-(6-aminohexyl)guanidine
(2E)-2-((2E)-2-{[amino(imino)methyl]hydrazono}-1-methylethylidene)hydrazinecarboximidamide
4-nitrobenzaldehyde [(2Z)-1-methylpyrimidin-2(1H)-ylidene]hydrazone
4-{[{[amino(imino)methyl]amino}(imino)methyl]amino}benzenesulfonic acid
N-1,3-benzodioxol-5-ylimidodicarbonimidic diamide
N,3-di-tert-butyl-3,4-dihydroquinazolin-2-amine
N-(4-fluorophenyl)-N′-(4-hydroxy-6-methylpyrimidin-2-yl)guanidine
N-(2-chloro-6-methylphenyl)imidodicarbonimidic diamide
N-hydroxyhydrazinecarboximidamide
(2S)-2-amino-6-{[imino(methylamino)methyl]amino}hexanoic acid 1H-perimidin-2-amine
N-[3-(1H-imidazol-4-yl)propyl]guanidine
N-(tert-butyl)-5H-dibenzo[d,f][1,3]diazepin-6-amine
N-(3-chloro-4-fluorophenyl)imidodicarbonimidic diamide
N-(3-chloro-2-fluorophenyl)imidodicarbonimidic diamide
(2-{[amino(imino)methyl]amino}-1,3-thiazol-4-yl)methylimidothiocarbamate
N-(tert-butyl)-N′,N″-diphenylguanidine
N-(1,2-dineopentyldiaziridin-3-ylidene)-N-neopentylamine
N, N′,N″-trineopentylguanidine
1-[bis(isopropylamino)methylene]-2-phenylhydrazine
N″-(benzyloxy)-N-(4,6-dimethylpyrimidin-2-yl)guanidine
N-(2-methyl-5-nitrophenyl)imidodicarbonimidic diamide
methyl 2-amino-6-{[amino(imino)methyl]amino}hexanoate
N-(4-hydroxy-6-methylpyrimidin-2-yl)-N′-(4-methoxyphenyl)guanidine
N-[4-hydroxy-6-(methoxymethyl)pyrimidin-2-yl]-N′-phenylguanidine
N-(4-hydroxy-6-methylpyrimidin-2-yl)-N′-(3-methoxyphenyl)guanidine
N-(4-hydroxy-6-methylpyrimidin-2-yl)-N′-(2-methoxyphenyl)guanidine
N,6-di-tert-butyl-8-methyl-3,4-dihydroquinazolin-2-amine
N-(3,5-dimethoxyphenyl)imidodicarbonimidic diamide
N-(3,4-dimethoxyphenyl)imidodicarbonimidic diamide
N-[2-(5-methoxy-1H-indol-3-yl)ethyl]imidodicarbonimidic diamide
4-{[amino(imino)methyl]amino}benzoic acid
N-(4-chlorophenyl)-N′-(4,6-dimethylpyrimidin-2-yl)guanidine
N,N′-di-tert-butyl-N″-[2-(hydroxymethyl)phenyl]guanidine
N-(4-chlorophenyl)-N′-(4-hydroxy-6-methylpyrimidin-2-yl)guanidine
N-(3-chlorophenyl)-N′-(4-hydroxy-6-methylpyrimidin-2-yl)guanidine
N-(2-chlorophenyl)-N′-(4-hydroxy-6-methylpyrimidin-2-yl)guanidine
N-(2,4-difluorophenyl)-N′-(4-hydroxy-6-methylpyrimidin-2-yl)guanidine
N-(2,6-difluorophenyl)-N′-(4-hydroxy-6-methylpyrimidin-2-yl)guanidine (3-benzyl-2-imino-2,3-dihydro-1H-benzimidazol-1-yl)acetic acid
N,N′-di-tert-butyl-N″-methylguanidine
N,N″-di-tert-butyl-N′-methylguanidine
N-[2-(trifluoromethyl)phenyl]imidodicarbonimidic diamide
N-[3-(trifluoromethyl)phenyl]imidodicarbonimidic diamide
N-[4-(trifluoromethyl)phenyl]imidodicarbonimidic diamide
N-(2,3-dichlorophenyl)imidodicarbonimidic diamide
N-(2,5-dichlorophenyl)imidodicarbonimidic diamide
N-(3,4-dichlorophenyl)imidodicarbonimidic diamide
N-(3,5-dichlorophenyl)imidodicarbonimidic diamide
N-(2,4-dichlorophenyl)imidodicarbonimidic diamide
4-{[[(4,6-dimethylpyrimidin-2-yl)amino](imino)methyl]amino}benzoic acid
9,13b-dihydro-1H-dibenzo[c,f]imidazo[1,5-a]azepin-3-amine
dipotassium [(carboxymethyl)(methyl)amino](imino)methylamidophosphate
N,N′,N″-triphenylguanidine
N-dodecylguanidine-N-(1,2-di-tert-butyidiaziridin-3-ylidene)-N-mesityiamine
N,3-di-tert-butyl-6,8-dimethyl-3,4-dihydroquinazolin-2-amine
N-(4-fluorophenyl)-N′-[3-hydroxy-5-(methoxymethyl)phenyl]guanidine
N,N′-di-tert-butyl-N″-mesitylguanidine
2-anilino-1,3-dimethyl-4,5-dihydro-1H-imidazole
N-(4-fluorophenyl)-N′-[4-hydroxy-6-(methoxymethyl)pyrimidin-2-yl]guanidine
N-(4-chlorophenyl)-N′-(4-hydroxy-5,6-dimethylpyrimidin-2-yl)guanidine
5-bromo-N4,5-dihydro-1H-imidazol-2-ylquinoxalin-6-amine
N-(4-bromophenyl)imidodicarbonimidic diamide
N,N′-dicyclohexylmorpholine-4-carboximidamide
5-[(4S)-2-iminohexahydro-1H-thieno[3,4-d]imidazol-4-yl]pentanohydrazide
N-(3-chloro-4-fluorophenyl)-N′-(4-hydroxy-6-methylpyrimidin-2-yl)guanidine
N2-(4-bromophenyl)-6,6-dimethyl-1,6-dihydro-1,3,5-triazine-2,4-diamine (2S)-2-amino-4-{[(hydroxyamino)(imino)methyl]amino}butanoic acid
N-(2-azocan-1-ylethyl)guanidine
{[(E)-[(4-methylbenzyl)amino](phenylimino)methyl]amino}acetic acid
N-[4-(trifluoromethoxy)phenyl]imidodicarbonimidic diamide
4-nitrophenyl 4-{[amino(imino)methyl]amino}benzoate
6-[(4-nitrobenzyl)sulfanyl]-1H-purin-2-amine
N,N′,2-triphenylhydrazinecarboximidamide
N-[4-hydroxy-6-(methoxymethyl)pyrimidin-2-yl]-N′-(3-methoxyphenyl)guanidine
N-[4-hydroxy-6-(methoxymethyl)pyrimidin-2-yl]-N′-(4-methoxyphenyl)guanidine
N-(3,4,5-trimethoxyphenyl)imidodicarbonimidic diamide
N-[(1E)-hydrazino(2-phenylhydrazino)methylene]benzenesulfonamide
N-[bis(neopentylamino)methylene]-2,2-dimethylpropan-1-amine
5-[3-({imino[(2,2,2-trifluoroethyl)amino]methyl}amino)-1H-pyrazole-1-yl]pentanamide
N-{3-[3-chloro-5-(trifluoromethyl)pyrid-2-yl]-1,2,4-oxadiazol-5-yl}guanidine
N-(4-chlorophenyl)-N′-[4-hydroxy-6-(methoxymethyl)pyrimidin-2-yl]guanidine
N-(2-chlorophenyl)-N′-[4-hydroxy-6-(methoxymethyl)pyrimidin-2-yl]guanidine
N-(2,4-difluorophenyl)-N′-[4-hydroxy-6-(methoxymethyl)pyrimidin-2-yl]guanidine
N,N′-di-tert-butyl-N″-isopropylguanidine
5,8-di-tert-butyl-N-methyl-1H-perimidin-2-amine
N-(4-hydroxy-6-methylpyrimidin-2-yl)-N′-[3-(trifluoromethyl)phenyl]guanidine
N-(4-hydroxy-6-methylpyrimidin-2-yl)-N′-[4-(trifluoromethyl)phenyl]guanidine
N-(3,4-dichlorophenyl)-N′-(4-hydroxy-6-methylpyrimidin-2-yl)guanidine
N-(3,5-dichlorophenyl)-N′-(4-hydroxy-6-methylpyrimidin-2-yl)guanidine
N-(2,3-dichlorophenyl)-N′-(4-hydroxy-6-methylpyrimidin-2-yl)guanidine
1-[bis(isopropylamino)methylene]-1-isopropyl-2-phenylhydrazine
N-{(5Z)₄-oxo-5-[2-(1H-1,2,4-triazol-1-yl)benzylidene]-4,5-dihydro-1,3-thiazol-2-yl}guanidine
N-(4-hydroxy-5,6,7,8-tetrahydroquinazolin-2-yl)-N′-(3-methoxyphenyl)guanidine
N-[(2Z)-1-methyl-4-phenylquinazolin-2(1H)-ylidene]methanesulfonamide
N-(1,3-dimethyl-1,3-dihydro-2H-benzimidazol-2-ylidene)-4-methylbenzenesulfonamide
N-(4-chlorophenyl)-N′-(4-hydroxy-5,6,7,8-tetrahydroquinazolin-2-yl)guanidine
N-(2,4-difluorophenyl)-N′-(4-hydroxy-5,6,7,8-tetrahydroquinazolin-2-yl)guanidine
N-(4-tert-butyl-6-hydroxypyrimidin-2-yl)-N′-(4-chlorophenyl)guanidine
N-(4-tert-butyl-6-hydroxypyrimidin-2-yl)-N′-(2,4-difluorophenyl)guanidine
ethyl 4-[(6-{[amino(imino)methyl]amino}hexanoyl)oxy]benzoate
N-(tert-butyl)-N′,N″-bis(2,6-dimethylphenyl)guanidine
N,N-bis(2-chlorobenzyl)-N″-hydroxyguanidine (2S)-6-{[amino(imino)methyl]amino}-2-[(tert-butoxycarbonyl)amino]hexanoic acid
N-(3-chloro-4-fluorophenyl)-N′-[4-hydroxy-6-(methoxymethyl)pyrimidin-2-yl]guanidine
N-(3-bromobenzyl)guanidine
N-(4-hydroxy-6-methylpyrimidin-2-yl)-N′-[4-(trifluoromethoxy)phenyl]guanidine
N-(4-tert-butyl-2,6-dimethylphenyl)-N-(1,2-di-tert-butyidiaziridin-3-ylidene)amine
N-(2-{[amino(imino)methyl]amino}ethyl)isoquinoline-5-sulfonamide
N-[4-hydroxy-6-(methoxymethyl)pyrimidin-2-yl]-N′-[3-(trifluoromethyl)phenyl]guanidine
N-(3,4-dichlorophenyl)-N′-[4-hydroxy-6-(methoxymethyl)pyrimidin-2-yl]guanidine
N-(3,5-dichlorophenyl)-N′-[4-hydroxy-6-(methoxymethyl)pyrimidin-2-yl]guanidine
N-(tert-butyl)-N′-mesityl-N″-methylguanidine
N,N″-di-tert-butyl-N′-phenylguanidine
(2S)-2-[(tert-butoxycarbonyl)amino]-6-{[(diethylamino) (imino)methyl]amino}hexanoic acid
(2R)-2-[(tert-butoxycarbonyl)amino]-6-{[(diethylamino) (imino)methyl]amino}hexanoic acid
N-[3,5-bis(trifluoromethyl)phenyl]imidodicarbonimidic diamide
3-(acetylamino)-4-{[amino(imino)methyl]amino}-2-(1,2,3-trihydroxypropyl)-3,4-dihydro-2H-pyran-6-carboxylic acid
piperazine-1-carboximidamide
N-{8-[(8-{[amino(imino)methyl]amino}octyl)amino]octyl}guanidine
N-[4-hydroxy-6-(methoxymethyl)pyrimidin-2-yl]-N′-[4-(trifluoromethoxy)phenyl]guanidine
N-{2-[(2-{[amino(imino)methyl]amino}ethyl)disulfanyl]ethyl}guanidine
N-(4-methylphenyl)guanidine
N-[amino(imino)methyl]-4-{[(1E)-3-(4-fluorophenyl)-3-oxoprop-1-enyl]amino}benzenesulfonamide
4-benzyl-N-(3,5-dichlorophenyl)piperazine-1-carboximidamide
N″-(tert-butyl)-N,N′-diphenylguanidine
N-(tert-butyl)-N′-(2,6-diisopropyl-4-phenoxyphenyl)guanidine
N,N″-di-tert-butyl-N′-(2,6-dimethylphenyl)guanidine
N,N′,N″-tris(2,6-dimethylphenyl)guanidine
(2S)-5-{[amino(imino)methyl]amino}-2-{[(benzoylamino)acetyl]oxy}pentanoic acid
4-methyl-2-oxo-2H-chromen-7-yl 4-{[amino(imino)methyl]amino}benzoate
N-(2,6-dichlorophenyl)-N-(4-fiuorobenzyl)-4,5-dihydro-1H-imidazol-2-amine
4-methylpiperazine-1-carboximidamide
N-[2-(benzhydryloxy)ethyl]guanidine
N-benzylguanidine
N,N″-di-tert-butyl-N′-(4-tert-butylphenyl)guanidine
N-(2-chlorophenyl)guanidine
N-(2-chloroethyl)-N-[3,5-dichloro-4-(imidazolidin-2-ylideneamino)benzyl]-N-methylamine
barium 2-(5-{[amino(imino)methyl]amino}-2-hydroxypentanoyl)succinate
N,N″-di-tert-butyl-N′-(4-tert-butyl-2-methylphenyl)guanidine
tert-butyl(1S)-1-(hydroxymethyl)-4-[(imino{[(4-methyl phenyl)sulfonyl]amino}methyl)amino]butylcarbamate
N-{8-[(8-{[amino(imino)methyl]amino}octyl)amino]octyl}guanidine
ethyl 4-[(6-{[amino(imino)methyl]amino}hexanoyl)oxy]benzoate
N″-(tert-butyl)-N,N′-bis(2,6-dimethylphenyl)guanidine
7-{[amino(imino)methyl]amino}-N-[2-({4-[(3-aminopropyl)amino]butyl}amino)-1-hydroxy-2-oxoethyl]heptanamide
N,N″-di-tert-butyl-N′-(4-tert-butyl-2,6-dimethylphenyl)guanidine
N-octylguanidine
3,4-dihydroisoquinoline-2(1H)-carboximidamide
N-(2,5-dimethoxyphenyl)guanidine
[[imino(phosphonoamino)methyl](methyl)amino]acetic acid
N-(2-{[(E)-(tert-butylamino)(tert-butylimino)methyl]amino}benzyl)-N,N-diethylethanamine
N-[3-(trifluoromethyl)phenyl]guanidine
N-{bis[(2,6-dimethylphenyl)amino]methylene}-2,6-dimethylbenzeneamine
4-{2-[2-(dimethylamino)-2-oxoethoxy]-2-oxoethyl}phenyl 4-{[amino(imino)methyl]amino}benzoate
N-(4-chlorophenyl)-N′-(6-{[{[[(4-chlorophenyl)amino](imino)methyl]amino}(imino)methyl]amino}hexyl)imidodicarbonimidic diamide
N-(2,3-dihydro-1,4-benzodioxin-2-ylmethyl)guanidine
N-(2-{[(E)-(tert-butylamino)(tert-butylimino)methyl]amino}benzyl)-N,N-diethylethanamine
6-[amino(imino)methyl]-2-naphthyl 4-{[amino(imino)methyl]amino}benzoate dimethanesulfonate
N,N′dicyclohexylguanidine
N-(4-chlorophenyl)-N′-(6-{[{[[(4-chlorophenyl)amino](imino)methyl]amino}(imino)methyl]amino}hexyl)imidodicarbonimidic diamide
N-(4-chlorophenyl)-N′-(6-{[{[[(4-chlorophenyl)amino](imino)methyl]amino}(imino)methyl]amino}hexyl)imidodicarbonimidic diamide
N-(2-ethylhexyl)-N′-(6-{[{[[(2-ethylhexyl)amino](imino)methyl]amino}(imino)methyl]amino}hexyl)imidodicarbonimidic diamide
n-propylguanidine
isopropylguanidine
n-butylguanidine
(2-methylpropyl)guanidine
t-butylguanidine
n-pentylguanidine
n-hexylguanidine
n-octylguanidine
(2-ethylhexyl)guanidine
n-decylguanidine
n-dodecylguanidine
(2-hydroxyethyl)guanidine
(3-hydroxypropyl)guanidine
(2-hydroxypropyl)guanidine
(4-hydroxybutyl)guanidine
(5-hydroxypentyl)guanidine
(6-hydroxyhexyl)guanidine
(1-hydroxymethylpentyl)guanidine
(2-methoxyethyl)guanidine
(2-ethoxyethyl)guanidine
(3-methoxypropyl)guanidine
(3-ethoxypropyl)guanidine
(3-propyloxypropyl)guanidine
(3-butyloxypropyl)guanidine
(3-pentyloxypropyl)guanidine
(3-hexyloxypropyl)guanidine
guanidinoacetic acid
3-guanidinopropanoic acid
4-guanidinobutanoic acid
5-guanidinopentanoic acid
6-guanidinohexanoic acid
methyl guanidinoacetate
ethyl guanidinoacetate
propyl guanidinoacetate
methyl 3-guanidinopropanoate
ethyl 3-guanidinopropanoate
isopropyl 3-guanidinopropanoate
methyl 4-guanidinobutanoate
ethyl 4-guanidinobutanoate
methyl 5-guanidinopentanoate
ethyl 5-guanidinopentanoate
methyl 6-guanidinohexanoate
ethyl 6-guanidinohexanoate
isopropyl 6-guanidinohexanoate
o-, m- or p-hydroxyphenylguanidine
o- or m-methylphenylguanidine
o-, m- or p-methoxyphenylguanidine
o-, m- or p-ethoxyphenylguanidine
o-, m- or p-guanidinobenzoic acid
methyl o-, m- or p-guanidinobenzoate
ethyl o-, m- or p-guanidinobenzoate
o-, m- or p-guanidinophenyl acetate
o-, m- or p-guanidinophenyl propanoate
o-, m- or p-guanidinophenylacetic acid
o-, m- or p-guanidinophenylpropanoic acid
o-, m- or p-guanidinophenylbutanoic acid
o-, m- or p-guanidinophenylpentanoic acid
o-, m- or p-guanidinophenylhexanoic acid
phenethylguanidine
naphthylguanidine
o-, m- or p-hydroxybenzylguanidine
o-, m- or p-methylbenzylguanidine
o-, m- or p-methoxybenzylguanidine
o-, m- or p-ethoxybenzylguanidine
o-, m- or p-guanidinomethylbenzoic acid
methyl o-, m- or p-guanidinomethylbenzoate
ethyl o-, m- or p-guanidinomethylbenzoate
isopropyl o-, m- or p-guanidinomethylbenzoate
cyclopentylguanidine
cyclohexylguanidine
cyclohexylmethylguanidine
4-guanidinomethylcyclohexanecarboxylic acid
2-guanidinocyclohexanecarboxylic acid
3-guanidinocyclohexanecarboxylic acid
4-guanidinocyclohexanecarboxylic acid
methyl 2-guanidinocyclohexanecarboxylate
ethyl 2-guanidinocyclohexanecarboxylate
4-oxacyclohexylguanidine
glucopyranosylguanidine
deoxyguanidinoinositol
2-(2-hydroxyethoxy)ethylguanidine
2-[2-(2-hydroxy)ethoxy]ethylguanidine
2-(2-methoxyethoxy)ethylguanidine
2-(2-ethoxyethoxy)ethylguanidine
2-[2-(2-methoxyethoxy)ethyl]guanidine
2-[2-(2-ethoxyethoxy)ethoxy]ethylguanidine
2-guanidinoethanesulfonic acid
3-guanidinopropanesulfonic acid
4-guanidinobutanesulfonic acid
5-guanidinopentanesulfonic acid
6-guanidinohexanesulfonic acid
3-guanidino-2-hydroxy-1-propanesulfonic acid
2-guanidinoethyl dihydrogenophosphate
3-guanidinopropyl dihydrogenophosphate
4-guanidinobutyl dihydrogenophosphate
5-guanidinopentyl dihydrogenophosphate
6-guanidinohexyl dihydrogenophosphate
biguanido(guanylguanidine)
1-methylbiguanide
1-ethylbiguanide
1-n-propylbiguanide
1-isopropylbiguanide
1-n-butylbiguanide
1-(2-methylpropyl)biguanide
1-t-butylbiguanide
1-n-pentylbiguanide
1-n-hexylbiguanide
1-n-octylbiguanide
1-(2-ethylhexyl)biguanide
1-n-decylbiguanide
1-n-dodecylbiguanide
1-(2-hydroxyethyl)biguanide
1-(3-hydroxypropyl)biguanide
1-(2-hydroxypropyl)biguanide
1-(4-hydroxybutyl)biguanide
1-(5-hydroxypentyl)biguanide
1-(6-hydroxyhexyl)biguanide
1-(1-hydroxymethylpentyl)biguanide
1-(2-methoxyethyl)biguanide
1-(2-ethoxyethyl)biguanide
1-(3-methoxypropyl)biguanide
1-(3-ethoxypropyl)biguanide
1-(3-propyloxypropyl)biguanide
1-(3-butyloxypropyl)biguanide
1-(3-pentyloxypropyl)biguanide
1-(3-hexyloxypropyl)biguanide
1-phenylbiguanide
1-(o-, m- or p-hydroxyphenyl)biguanide
1-(o-, m- or p-methylphenyl)biguanide
o-, m- or p-methoxyphenylbiguanide
o-, m- or p-ethoxyphenylbiguanide
1-benzylbiguanide
1-cyclopentylbiguanide
1-cyclohexylbiguanide
1-(4-oxacyclohexyl)biguanide
1-[2-(2-hydroxyethoxy)ethyl]biguanide
1-[2-[2-(2-hydroxyethoxy)ethoxy]ethyl]biguanide
1,1-dimethylbiguanide
1,1-diethylbiguanide
1-ethyl-1-methylbiguanide
1,1-dipropylbiguanide
1,1-dibutylbiguanide
1,1-di-n-propylguanidine
1,1-diisopropylguanidine
1,1-di-n-butylguanidine
1,1-bis(2-methylpropyl)guanidine
1,1-di-t-butylguanidine
1,1-di-n-pentylguanidine
1,1-bis(2-ethylhexyl)guanidine
1,1-di-n-hexylguanidine
1,1-dioctylguanidine
1,1-di-n-decylguanidine
1,1-di-n-dodecylguanidine
1-methyl-1-ethylguanidine
1-methyl-1-n-propylguanidine
1-methyl-1-isopropylguanidine
1-methyl-1-n-butylguanidine
1-methyl-1-(2-methylpropyl)guanidine
1-methyl-1-t-butylguanidine
1-methyl-1-n-pentylguanidine
1-methyl-1-(2-ethylhexyl)guanidine
1-methyl-1-n-hexylguanidine
1-methyl-1-n-octylguanidine
1-methyl-1-n-decylguanidine
1-methyl-1-n-dodecylguanidine
1,1-bis(2-hydroxyethyl)guanidine
1,1-bis(3-hydroxypropyl)guanidine
1,1-bis(2-hydroxypropyl)guanidine
1,1-bis(4-hydroxybutyl)guanidine
1,1-bis(5-hydroxypentyl)guanidine
1,1-bis(6-hydroxyhexyl)guanidine
1,1-bis(1-hydroxymethylpentyl)guanidine
1-methyl-1-(2-hydroxyethyl)guanidine
1-methyl-1-(3-hydroxypropyl)guanidine
1-methyl-1-(2-hydroxypropyl)guanidine
1-methyl-1-(4-hydroxybutyl)guanidine
1-methyl-1-(5-hydroxypentyl)guanidine
1-methyl-1-(6-hydroxyhexyl)guanidine
1-methyl-1-(1-hydroxymethylpentyl)guanidine
1,1-bis(2-methoxyethyl)guanidine
1,1-bis(2-ethoxyethyl)guanidine
1,1-bis(3-methoxypropyl)guanidine
1,1-bis(3-ethoxypropyl)guanidine
1,1-bis(3-propyloxypropyl)guanidine
1,1-bis(3-butyloxypropyl)guanidine
1,1-bis(3-pentyloxypropyl)guanidine
1,1-bis(3-hexyloxypropyl)guanidine
1-methyl-1-(2-methoxyethyl)guanidine
1-methyl-1-(2-ethoxyethyl)guanidine
1-methyl-1-(3-methoxypropyl)guanidine
1-methyl-1-(3-ethoxypropyl)guanidine
1-methyl-1-(3-propyloxypropyl)guanidine
1-methyl-1-(3-butyloxypropyl)guanidine
1-methyl-1-(3-pentyloxypropyl)guanidine
1-methyl-1-(3-hexyloxypropyl)guanidine
1-methylguanidinoacetic acid
3-(1-methylguanidino)propanoic acid
4-(1-methylguanidino)butanoic acid
5-(1-methylguanidino)pentanoic acid
6-(1-methylguanidino)hexanoic acid
methyl 1-methylguanidinoacetate
ethyl 1-methylguanidinoacetate
propyl 1-methylguanidinoacetate
methyl 3-(1-methylguanidino)propanoate
ethyl 3-(1-methylguanidino)propanoate
propyl 3-(1-methylguanidino)propanoate
isopropyl 3-(1-methylguanidino)propanoate
methyl 4-(1-methylguanidino)butanoate
methyl 5-(1-methylguanidino)pentanoate
ethyl 5-(1-methylguanidino)pentanoate
methyl 6-(1-methylguanidino)hexanoate
ethyl 6-(1-methylguanidino)hexanoate
1,1-diphenylguanidine
1,1-bis(o-, m- or p-hydroxyphenyl)guanidine
1,1-bis(o-, m- or p-methylphenyl)guanidine
1,1-bis(o-, m- or p-methoxyphenyl)guanidine
1,1-bis(o-, m- or p-ethoxyphenyl)guanidine
1-methyl-1-phenylguanidine
1-methyl-1-(o-, m- or p-hydroxyphenyl)guanidine
1-methyl-1-(o-, m- or p-methylphenyl)guanidine
1-methyl-1-(o-, m- or p-methoxyphenyl)guanidine
1-methyl-1-(o-, m- or p-ethoxyphenyl)guanidine
o-, m- or p-(1-methylguanidino)benzoic acid
methyl o-, m- or p-(1-methylguanidino)benzoate
ethyl o-, m- or p-(1-methylguanidino)benzoate
o-, m- or p-(1-methylguanidino)phenyl acetate
o-, m- or p-(1-methylguanidino)phenyl propanoate
o-, m- or p-(1-methylguanidino)phenylacetic acid
o-, m- or p-(1-methylguanidino)phenylpropanoic acid
o-, m- or p-(1-methylguanidino)phenylbutanoic acid
o-, m- or p-(1-methylguanidino)phenylpentanoic acid
o-, m- or p-(1-methylguanidino)phenylhexanoic acid
1,1-dibenzylguanidine
1,1-bis[o-, m- or p-(hydroxybenzyl)]guanidine
1,1-bis[o-, m- or p-(methylbenzyl)]guanidine
1,1-bis[o-, m- or p-(methoxybenzyl)]guanidine
1,1-bis[o-, m- or p-(ethoxybenzyl)]guanidine
1-methyl-1-benzylguanidine
1-methyl-1-[o-, m- or p-(hydroxybenzyl)]guanidine
1-methyl-1-[o-, m- or p-(methylbenzyl)]guanidine
1-methyl-1-[o-, m- or p-(methoxybenzyl)]guanidine
1-methyl-1-[o-, m- or p-(ethoxybenzyl)]guanidine
o-, m- or p-(1-methylguanidinomethyl)benzoic acid
methyl o-, m- or p-(1-methylguanidinomethyl)benzoate
ethyl o-, m- or p-(1-methylguanidinomethyl)benzoate
isopropyl o-, m- or p-(1-methylguanidinomethyl)benzoate
1,1-dicyclopentylguanidine
1,1-dicyclohexylguanidine
1-methyl-1-cyclopentylguanidine
1-methyl-1-cyclohexylguanidine
2-(1-methylguanidino)cyclohexanecarboxylic acid
3-(1-methylguanidino)cyclohexanecarboxylic acid
4-(1-methylguanidino)cyclohexanecarboxylic acid
methyl 2-(1-methylguanidino)cyclohexanecarboxylate
ethyl 2-(1-methylguanidino)cyclohexanecarboxylate
1,1-bis(4-oxacyclohexyl)guanidine
1,1-di(glucopyranosyl)guanidine
1-methyl-1-(4-oxacyclohexyl)guanidine
1-methyl-1-(glucopyranosyl)guanidine
1,1-bis[2-(2-hydroxyethoxy)ethyl]guanidine
1,1-bis[2-[2-(2-hydroxyethoxy)ethoxy]ethyl]guanidine
1-methyl-1-[2-(2-hydroxyethoxy)ethyl]guanidine
1-methyl-1-[2-(2-hydroxyethoxy)ethoxy]ethylguanidine
1,1-bis[2-(2-methoxyethoxy)ethyl]guanidine
1,1-bis[2-(2-ethoxyethoxy)ethyl]guanidine
1,1-bis[2-[2-(2-methoxyethoxy)ethoxy]ethyl]guanidine
1,1-bis[2-[2-(2-ethoxyethoxy)ethoxy]ethyl]guanidine
1-methyl-1-[2-(2-methoxyethoxy)ethyl]guanidine
1-methyl-1-[2-(2-ethoxyethoxy)ethyl]guanidine
1-methyl-1-[2-[2-(2-methoxyethoxy)ethoxy]ethyl]guanidine
1-methyl-1-[2-[2-(2-ethoxyethoxy)ethoxy]ethyl]guanidine
1,3-dimethylguanidine
1,3-di-n-propylguanidine
1,3-di-n-butylguanidine
1,3-bis(2-methylpropyl)guanidine
1,3-di-t-butylguanidine
1,3-di-n-pentylguanidine
1,3-bis(2-ethylhexyl)guanidine
1,3-di-n-hexylguanidine
1-dioctylguanidine
1,3-di-n-decylguanidine
1,3-di-n-dodecylguanidine
3-methyl-1-ethylguanidine
3-methyl-1-n-propylguanidine
3-methyl-1-isopropylguanidine
3-methyl-1-n-butylguanidine
3-methyl-1-(2-methylpropyl)guanidine
3-methyl-1-t-butylguanidine
3-methyl-1-n-pentylguanidine
3-methyl-1-(2-ethylhexyl)guanidine
3-methyl-1-n-hexylguanidine
3-methyl-1-n-octylguanidine
3-methyl-1-n-decylguanidine
3-methyl-1-n-dodecylguanidine
1,3-bis(2-hydroxyethyl)guanidine
1,3-bis(3-hydroxypropyl)guanidine
1,3-bis(2-hydroxypropyl)guanidine
1,3-bis(4-hydroxybutyl)guanidine
1,3-bis(5-hydroxypentyl)guanidine
1,3-bis(6-hydroxyhexyl)guanidine
1,3-bis(1-hydroxymethylpentyl)guanidine
3-methyl-1-(2-hydroxyethyl)guanidine
3-methyl-3-hydroxypropyl)guanidine
3-methyl-1-(2-hydroxypropyl)guanidine
3-methyl-1-(4-hydroxybutyl)guanidine
3-methyl-1-(5-hydroxypentyl)guanidine
3-methyl-1-(6-hydroxyhexyl)guanidine
3-methyl-1-(hydroxymethylpentyl)guanidine
1,3-bis(2-methoxyethyl)guanidine
1,3-bis(2-ethoxyethyl)guanidine
1,3-bis(3-methoxypropyl)guanidine
1,3-bis(3-ethoxypropyl)guanidine
1,3-bis(3-propyloxypropyl)guanidine
1,3-bis(3-butyloxypropyl)guanidine
1,3-bis(3-pentyloxypropyl)guanidine
1,3-bis(3-hexyloxypropyl)guanidine
3-methyl-1-(2-methoxyethyl)guanidine
3-methyl-1-(2-ethoxyethyl)guanidine
3-methyl-1-(3-methoxypropyl)guanidine
3-methyl-1-(3-ethoxypropyl)guanidine
3-methyl-1-(3-propyloxypropyl)guanidine
3-methyl-1-(3-butyloxypropyl)guanidine
3-methyl-1-(3-pentyloxypropyl)guanidine
3-methyl-1-(3-hexyloxypropyl)guanidine
3-methylguanidinoacetic acid
3-(3-methylguanidino)propanoic acid
4-(3-methylguanidino)butanoic acid
5-(3-methylguanidino)pentanoic acid
6-(3-methylguanidino)hexanoic acid
methyl 3-methylguanidinoacetate
ethyl 3-methylguanidinoacetate
propyl 3-methylguanidinoacetate
methyl 3-(3-methylguanidino)propanoate
ethyl 3-(3-methylguanidino)propanoate
propyl 3-(3-methylguanidino)propanoate
isopropyl 3-(3-methylguanidino)propanoate
methyl 4-(3-methylguanidino)butanoate
ethyl 4-(3-methylguanidino)butanoate
propyl 4-(3-methylguanidino)butanoate
isopropyl 4-(3-methylguanidino)butanoate
methyl 5-(3-methylguanidino)pentanoate
ethyl 5-(3-methylguanidino)pentanoate
methyl 6-(3-methylguanidino)hexanoate
ethyl 6-(3-methylguanidino)hexanoate
1,3-bis(o-, m- or p-hydroxyphenyl)guanidine
1,3-bis(m- or p-methylphenyl)guanidine
1,3-bis(o-, m- or p-methoxyphenyl)guanidine
1,3-bis(o-, m- or p-ethoxyphenyl)guanidine
3-methyl-1-phenylguanidine
3-methyl-1-(o-, m- or p-hydroxyphenyl)guanidine
3-methyl-1-(o-, m- or p-methylphenyl)guanidine
3-methyl-1-(o-, m- or p-methoxyphenyl)guanidine
3-methyl-1-(o-, m- or p-ethoxyphenyl)guanidine
o-, m- or p-(3-methylguanidino)benzoic acid
methyl o-, m- or p-(3-methylguanidino)benzoate
ethyl o-, m- or p-(3-methylguanidino)benzoate
o-, m- or p-(3-methylguanidino)phenyl acetate
o-, m- or p-(3-methylguanidino)phenyl propanoate
o-, m- or p-(3-methylguanidino)phenylacetic acid
o-, m- or p-(3-methylguanidino)phenylpropanoic acid
o-, m- or p-(3-methylguanidino)phenylbutanoic acid
o-, m- or p-(3-methylguanidino)phenylpentanoic acid
o-, m- or p-(3-methylguanidino)phenylhexanoic acid
1,3-dibenzylguanidine
1,3-bis[o-, m- or p-(hydroxybenzyl)]guanidine
1,3-bis[o-, m- or p-(methylbenzyl)]guanidine
1,3-bis[o-, m- or p-(methoxybenzyl)]guanidine
1,3-bis[o-, m- or p-(ethoxybenzyl)]guanidine
3-methyl-1-benzylguanidine
3-methyl-1-o-, m- or p-(hydroxybenzyl)guanidine
3-methyl-1-o-, m- or p-(methylbenzyl)guanidine
3-methyl-1-o-, m- or p-(methoxybenzyl)guanidine
3-methyl-1-o-, m- or p-(ethoxybenzyl)guanidine
o-, m- or p-(3-methylguanidinomethyl)benzoic acid
methyl o-, m- or p-(3-methylguanidinomethyl)benzoate
ethyl o-, m- or p-(3-methylguanidinomethyl)benzoate
isopropyl o-, m- or p-(3-methylguanidinomethyl)benzoate
1,3-dicyclopentylguanidine
1,3-dicyclohexylguanidine
3-methyl-1-cyclopentylguanidine
3-methyl-1-cyclohexylguanidine
2-(3-methylguanidino)cyclohexanecarboxylic acid
3-(3-methylguanidino)cyclohexanecarboxylic acid
4-(3-methylguanidino)cyclohexanecarboxylic acid
methyl 2-(3-methylguanidino)cyclohexanecarboxylate
ethyl 2-(3-methylguanidino)cyclohexanecarboxylate
1,3-bis(4-oxacyclohexyl)guanidine
1,3-bis(glucopyranosyl)guanidine
3-methyl-1-(4-oxacyclohexyl)guanidine
3-methyl-1-(glucopyranosyl)guanidine
1,3-bis[2-(2-hydroxyethoxy)ethyl]guanidine
1,3-bis[2-[2-(2-hydroxyethoxy)ethoxy]ethyl]guanidine
3-methyl-1-[2-(2-hydroxyethoxy)ethyl]guanidine
3-methyl-1-[2-(2-hydroxyethoxy)ethoxy]ethylguanidine
1,3-bis[2-(2-methoxyethoxy)ethyl]guanidine
1,3-bis[2-(2-ethoxyethoxy)ethyl]guanidine
1,3-bis[2-[2-(2-methoxyethoxy)ethoxy]ethyl]guanidine
1,3-bis[2-[2-(2-methoxymethoxy)ethoxy]ethyl]guanidine
3-methyl-1-[2-(2-methoxyethoxy)ethyl]guanidine
3-methyl-1-[2-(2-ethoxyethoxy)ethyl]guanidine
3-methyl-1-[2-[2-(2-methoxyethoxy)ethoxy]ethyl]guanidine
3-methyl-1-[2-[2-(2-ethoxyethoxy)ethoxy]ethyl]guanidine
N-[bis(dimethylamino)methylene]-N′,N″-diisopropylguanidine
   b) In some embodiments, the guanidine-type imines may be chosen from:
N,N-dimethylguanidine
N,N′,N″-trimethylguanidine
N-methylguanidine
N,N-diethylguanidine
N-ethylguanidine
N,N-dimethylimidodicarbonimidic diamide
N-(tert-butyl)-N″-methylguanidine
N-(4-aminobutyl)guanidine
1,3,4,6,7,8-hexahydro-2H-pyrimidol[1,2-a]pyrimidine
N,N″-diisopropylguanidine
1-methyl-1,3,4,6,7,8-hexahydro-2H-pyrimidol[1,2a]pyrimidine
N″-(tert-butyl)-N,N,N′,N′-tetramethylguanidine
N,N,N′,N′-tetraethylguanidine
N,N″-di-tert-butylguanidine
N″-[amino(imino)methyl]-N-phenylguanidine
N,N′,N″-triisopropylguanidine
pyrrolidine-1-carboximidamide
N-phenylguanidine
N-(tert-butyl)-N′,N″-dimethylguanidine
N-(4-{[amino(imino)methyl]amino}butyl)guanidine
N″-(tert-butyl)-N-methyl-N′-phenylguanidine
N, N′,N″-tricyclohexylguanidine
N-{3-[(4{[amino(imino)methyl]amino}butyl)amino]propyl}guanidine
N-(2-hydroxyethyl)-N-methylguanidine
N-(10-{[amino(imino)methyl]amino}decyl)guanidine
n-propylguanidine
isopropylguanidine
n-butylguanidine
(2-methylpropyl)guanidine
t-butylguanidine
n-pentylguanidine
n-hexylguanidine
(2-ethylhexyl)guanidine
(2-hydroxyethyl)guanidine
(3-hydroxypropyl)guanidine
(2-hydroxypropyl)guanidine
(4-hydroxybutyl)guanidine
(5-hydroxypentyl)guanidine
(6-hydroxyhexyl)guanidine
(1-hydroxymethylpentyl)guanidine
(2-methoxyethyl)guanidine
(2-ethoxyethyl)guanidine
(3-methoxypropyl)guanidine
(3-ethoxypropyl)guanidine
o-, m- or p-hydroxyphenylguanidine
o-, m-methylphenylguanidine
o-, m- or p-methoxyphenylguanidine
phenethylguanidine
o-, m- or p-hydroxybenzylguanidine
o-, m- or p-methylbenzylguanidine
o-, m- or p-methoxybenzylguanidine
cyclopentylguanidine
cyclohexylguanidine
cyclohexylmethylguanidine
4-oxacyclohexylguanidine
glucopyranosylguanidine
deoxyguanidinoinositol
2-(2-hydroxyethoxy)ethylguanidine
2-[2-(2-hydroxy)ethoxy]ethylguanidine
2-(2-methoxyethoxy)ethylguanidine
2-(2-ethoxyethoxy)ethylguanidine
2-[2-(2-methoxyethoxy)ethyl]guanidine
2-[2-(2-ethoxyethoxy)ethoxyl]ethylguanidine
biguanido(guanylguanidine)
1-methylbiguanide
1-ethylbiguanide
1-n-propylbiguanide
1-isopropylbiguanide
1-n-butylbiguanide
1-(2-methylpropyl)biguanide
1-t-butylbiguanide
1-n-pentylbiguanide
1-n-hexylbiguanide
1-(2-hydroxyethyl)biguanide
1-(3-hydroxypropyl)biguanide
1-(2-hydroxypropyl)biguanide
1-(4-hydroxybutyl)biguanide
1-phenylbiguanide
1-benzylbiguanide
1-cyclopentylbiguanide
1-cyclohexylbiguanide
14-oxacyclohexylbiguanide
1,1-dimethylbiguanide
1,1-diethylbiguanide
1-ethyl-1-methylbiguanide
1,1-di-n-propylguanidine
1,1-diisopropylguanidine
1,1-di-n-butylguanidine
1,1-bis(2-methylpropyl)guanidine
1,1-di-t-butylguanidine
1-methyl-1-ethylguanidine
1-methyl-1-n-propylguanidine
1-methyl-1-isopropylguanidine
1-methyl-1-n-butylguanidine
1,1-bis(2-hydroxyethyl)guanidine
1,1-bis(3-hydroxypropyl)guanidine
1,1-bis(2-hydroxypropyl)guanidine
1-methyl-1-(2-hydroxyethyl)guanidine
1-methyl-1-(3-hydroxypropyl)guanidine
1-methyl-1-(2-hydroxypropyl)guanidine
1,1-diphenylguanidine
1-methyl-1-phenylguanidine
1,1-dibenzylguanidine
1-methyl-1-benzylguanidine
1,3-dimethylguanidine
1,3-di-n-propylguanidine
1,3-di-n-butylguanidine
1,3-bis(2-methylpropyl)guanidine
1,3-di-t-butylguanidine
1,3-di-n-pentylguanidine
1,3-bis(2-ethylhexyl)guanidine
1,3-di-n-hexylguanidine
3-methyl-1-ethylguanidine
3-methyl-1-n-propylguanidine
3-methyl-1-isopropylguanidine
3-methyl-1-n-butylguanidine
3-methyl-1-(2-methylpropyl)guanidine
3-methyl-1-t-butylguanidine
3-methyl-1-n-pentylguanidine
1,3-bis(2-hydroxyethyl)guanidine
1,3-bis(3-hydroxypropyl)guanidine
1,3-bis(2-hydroxypropyl)guanidine
3-methyl-1-(2-hydroxyethyl)guanidine
3-methyl-(3-hydroxypropyl)guanidine
3-methyl-1-(2-hydroxypropyl)guanidine
1,3-dibenzylguanidine
3-methyl-1-benzylguanidine
1,3-dicyclopentylguanidine
1,3-dicyclohexylguanidine
3-methyl-1-cyclopentylguanidine
3-methyl-1-cyclohexylguanidine
1,3-di-(4-oxacyclohexyl)guanidine
1,3-di-(glucopyranosyl)guanidine
3-methyl-1-(4-oxacyclohexyl)guanidine
3-methyl-1-(glucopyranosyl)guanidine
N-[bis(dimethylamino)methylene]-N′,N″-diisopropylguanidine
   c) As a further example, the guanidine-type imines may be chosen from:
N,N-dimethylguanidine
N,N′,N″-trimethylguanidine
N-methylguanidine
N,N-diethylguanidine
N-ethylguanidine
N,N-dimethylimidodicarbonimidic diamide
N-(tert-butyl)-N″-methylguanidine
N-(4-aminobutyl)guanidine
1,3,4,6,7,8-hexahydro-2H-pyrimido[1,2-a]pyrimidine
N,N″-diisopropylguanidine
1-methyl-1,3,4,6,7,8-hexahydro-2H-pyrimido[1,2-a]pyrimidine
N″-(tert-butyl)-N,N, N′,N′-tetramethylguanidine
N,N,N′,N′-tetraethylguanidine
N,N″-di-tert-butylguanidine
N″-[amino(imino)methyl]-N-phenylguanidine
N,N′,N″-triisopropylguanidine
pyrrolidine-1-carboximidamide
N-phenylguanidine
N-(tert-butyl)-N′,N″-dimethylguanidine
N-(4-{[amino(imino)methyl]amino}butyl)guanidine
N″-(tert-butyl)-N-methyl-N′-phenylguanidine
N,N′,N″-tricyclohexylguanidine
N-{3-[(4-{[amino(imino)methyl]amino}butyl)amino]propyl}guanidine
N-(2-hydroxyethyl)-N-methylguanidine
n-propylguanidine
isopropylguanidine
(2-methylpropyl)guanidine
(2-ethylhexyl)guanidine
(2-hydroxyethyl)guanidine
(2-methoxyethyl)guanidine
o-, m- or p-hydroxybenzylguanidine
cyclopentylguanidine
cyclohexylguanidine
cyclohexylmethylguanidine
4-oxacyclohexylguanidine
glucopyranosylguanidine
biguanido(guanylguanidine)
1-methylbiguanide
1-ethylbiguanide
1-n-propylbiguanide
1-isopropylbiguanide
1-(2-methylpropyl)biguanide
1-(2-hydroxyethyl)biguanide
1-phenylbiguanide
1-benzylbiguanide
1-cyclopentylbiguanide
1-cyclohexylbiguanide
1,1-dimethylbiguanide
1,1-diethylbiguanide
1-ethyl-1-methylbiguanide
1-methyl-1-ethylguanidine
1-methyl-1-n-propylguanidine
1-methyl-1-isopropylguanidine
1,1-bis(2-hydroxyethyl)guanidine
1-methyl-1-phenylguanidine
3-methyl-1-ethylguanidine
3-methyl-1-n-propylguanidine
3-methyl-1-isopropylguanidine
1,3-bis(2-hydroxyethyl)guanidine
1,3-dibenzylguanidine
3-methyl-1-benzylguanidine
1,3-dicyclopentylguanidine
3-methyl-1-cyclopentylguanidine
3-methyl-1-cyclohexylguanidine
1,3-di-(4-oxacyclohexyl)guanidine
3-methyl-1-(4-oxacyclohexyl)guanidine
3-methyl-1-(glucopyranosyl)guanidine
N-[bis(dimethylamino)methylene]-N′,N″-diisopropylguanidine
2) Amidine-type imines,
   a) such as:
(3,3-dimethyl-3H-indol-2-yl)methylamine
1,2,3,4,4a,5,6,7-octahydro-2,2,4a,7,7-pentamethylnaphthyridine
1,2,3,9-tetrahydropyrrolo[2,1-b]quinazolin-9-ol
1,2,3,9-tetrahydropyrrolo[2,1-b]quinazoline
1,3-benzodioxole-5-carboximidamide
1,3-benzodioxole-5-ethanimidamide
1,4-benzodioxan-1-carboxamidine
1-benzothiophene-3-carboximidamide
1-benzyl-2,4-dioxohexahydro-5-pyrimidinecarboximidamide
1-methyl-2-(2-thiophen-2-ylvinyl)-1,4,5,6-tetrahydro-pyrimidine
1-methyl-2,4-dioxohexahydro-5-pyrimidinecarboximidamide
1-methyl-2-imino-,2-dihydropyridine
1-methylquinolin-2(1H)-imine
1-piperidinepropanimidamide
2-(1H-1,2,4-triazol-1-yl)ethanimidamide
2-(2,2-dimethylpropyl)-4,5-dihydro-1H-imidazole
2-(2,3-dihydrobenzo[1,4]dioxin-2-yl)-4,5-dihydro-1H-imidazole
2-(2,3-dihydrobenzo[1,4]dioxin-2-yl)-4,5-dihydro-1H-imidazole
2-(2-benzofuranyl)-2-imidazoline
2-(2-chlorophenoxy)ethanimidamide
2-(4,5-dihydro-1H-imidazol-2-yl)-6-methoxypyrazine
2-(4,5-dihydro-1H-imidazol-2-yl)pyrazine
2-(4,5-dihydroimidazol-2-yl)quinoline
2-(4-carbamimidoylphenyl)-1H-indole-6-carboxamidine
2-(4-chlorophenoxy)acetamidine
2-(4-pyridyl)cycloformanizine
2,2-dimethylpropionamidine
2,4-dimethyl-2-imidazoline
2,4-dioxo-1-(4-pyridinylmethyl)hexahydro-5-pyrimidinecarboximidamide
2-amidinopyridine
2-amidinopyrimidine
2-amidinothiophene
2-amino-4(3H)-pyrimidone
2-amino-4-[2-(3-amino-4-carbamimidoylphenyl)vinyl]benzamidine
2-benzyl-2-imidazoline
2-carbamimidoylacetamide
2-cyclopropylacetamidine
2-ethyl-4-methylimidazoline
2-furancarboximidamide
2-imino-4,6-dimethylpiperidine
2-imino-4-methylpiperidine
2-iminopiperidine
2-methyl-1,4,5,6-tetrahydropyrimidine-4-carboxylic acid, sodium salt
2-methyl-2-imidazoline
2-methylthiazole-4-carboxamidine
2-n-propyl-2-imidazoline
2-phenoxyacetamidine
2-phenyl-2-imidazoline
2-phenylethanimidamide
2-thiophenecarboxamidine
3-(4,5-dihydro-1H-imidazol-2-yl)phenylamine
3-(aminoiminomethyl)benzoic acid, sodium salt
3-[2-(1-methyl-1,4,5,6-tetrahydropyrimidin-2-yl)-vinyl]phenol
3-amino-1H-isoindole
ethyl 3-amino-3-iminopropanoate
3-aminobenzamidine
3-aminomethylbenzamidine
3-butenamidine
3-chlorobenzamidine
3-chloropropanimidamide
3-furancarboximidamide
3-methylbenzenecarboximidamide
3-nitrobenzenecarboximidamide
3-thiophenecarboximidamide
4-(1-pyrrolidine)butyramidine
4′,6-diamidino-2-phenylindole
4-[5-(4-carbamimidoylphenoxy)pentyloxy]benzamidine
4-amidinoanisole
4-amidinobenzamide
4-amidinobenzoic acid, sodium salt
4-amidinopyridine
4-aminobenzamidine
4-aminomethylbenzamidine
4-anisamidine
4-chlorobenzamidine
4-chlorobenzene-1-carboximidamide
4-hydroxybenzamidine
4-methoxycarbonylbenzamidine
4-methyl benzamidine
4-pyridinecarboxamidine
5,5-dimethyl-2-phenyl-4,5-dihydro-1H-imidazole
6H-6-imino-(2,3,4,5-tetrahydropyrimido)[1,2-c]-[1,3]benzothiazine
amidinophenylpyruvic acid, sodium salt
aminoacetamidine dihydrobromide
benzamidine
benzyl(4,5-dihydro-1H-imidazol-2-ylmethyl)phenylamine
chloroacetamidine
cyclobutanecarboxamidine
cyclohexanecarboxamidine
cyclopentanecarboximidamide
cyclopropanecarboxamidine
cyclopropylcarbamidine
deoxypeganine
ethyl amidinoacetate
heptanimidamide
N-(3,4-dihydro-2H-pyrrol-5-yl)-N-phenylamine
N-(3-chlorophenyl)acetamidine
N,N′-bis[3-(4,5-dihydro-1H-imidazol-2-yl)phenyl]urea dipropanoate
N,N′-diphenylbenzamidine
N,N′-di-p-tolylacetamidine
N-phenylbenzamidine
p-aminobenzamidine
p-aminobenzamidine HCl
pyrazine-2-carboxamidine
tert-butylcarbamidine
tolazoline
trifluoroacetamidine
   b) In some embodiments, the amidine-type imines may be chosen from, for example:
1,2-dimethyl-1,4,5,6-tetrahydropyrimidine
1-methyl-2-(2-thiophen-2-ylvinyl)-1,4,5,6-tetrahydro-pyrimidine
2,3,4,6,7,8,9,10-octahydropyrimido[1,2-a]azepine
2,3,4,6,7,8-hexahydropyrrolo[1,2-a]pyrimidine
2-methylpropanimidamide
2-methylpropionamidine
3-amidinopyridine
3-pyridinecarboxamidine
6-(dibutylamino)-1,8-diazabicyclo[5.4.0]undec-7-ene
acetamidine
butyramidine
propionamidine.

In the compositions intended for processes for permanently shaping keratin fibers, the at least one imine not belonging to the hydroxide family may be present in a molar concentration ranging from 0.01 M to 4M, which corresponds to concentrations ranging from 0.1% to 80% by weight, relative to the total weight of the composition, such as concentrations ranging from 0.05M to 2M, which corresponds to concentrations ranging from 0.5% to 40% by weight, relative to the total weight of the composition.

The pH of the compositions may range from 9.6 to 14, such as from 11 to 13.

In some embodiments, the imine not belonging to the hydroxide family is the only permanent hair-shaping active agent in the composition.

The compositions may also comprise at least one known reducing agent, such as thioglycolic acid or thiolactic acid and ester and amide derivatives thereof, for example glyceryl monothioglycolate, cysteamine and its C1-C4 acyl derivatives such as N-acetylcysteamine, N-propionyl-cysteamine, cysteine, N-acetylcysteine, thiomalic acid, pantethine, 2,3-dimercaptosuccinic acid, sulfites and bisulfites of an alkali metal or alkaline-earth metal, the N-(mercaptoalkyl)-ω-hydroxyalkylamides described in Patent Application EP-A-354,835, the N-mono- or N,N-dialkylmercapto-4-butyramides described in Patent Application EP-A-368,763, the aminomercaptoalkylamides described in Patent Application EP-A-432,000, the N-(mercaptoalkyl)succinamic acid and N-(mercaptoalkyl)succinimide derivatives described in Patent Application EP-A-465,342, the alkylamino mercaptoalkylamides described in Patent Application EP-A-514,282, the azeotropic mixture of 2-hydroxypropyl thioglycolate and of (2-hydroxy-1-methyl)ethyl thioglycolate described in Patent Application FR-A-2,679,448, the mercaptoalkylaminoamides described in Patent Application FR-A-2,692,481, the N-mercaptoalkylalkanediamides described in Patent Application EP-A-653,202, and the formamidinesulfinic acid derivatives described in Patent Publication No. WO 02/39965.

When the compositions comprise at least one reducing agent, the agent is present in a maximum concentration of 20% by weight, for example, from 0.1% to 10% by weight, relative to the total weight of the composition.

The compositions may also comprise at least one known hydroxide, chosen for example from alkali metal, alkaline-earth metal, transition metal, and organic hydroxides, such as sodium hydroxide, potassium hydroxide, lithium hydroxide, rubidium hydroxide, cesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminum hydroxide, guanidinium hydroxide and quaternary ammonium hydroxides.

When the compositions comprise at least one hydroxide, the hydroxide may be present in a concentration ranging from 0.01% to 3.5% by weight, such as from 0.05% to 1.5% by weight, relative to the total weight of the composition.

In some embodiments, the compositions contain 0% of base belonging to the hydroxide family, such as alkali metal, alkaline-earth metal, transition metal, or organic hydroxides.

In some embodiments, the compositions also comprise at least one surfactant chosen from nonionic, anionic, cationic and amphoteric surfactants, such as alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters and other hydroxypropyl ether nonionic surfactants.

When the basic compositions comprise at least one surfactant, the surfactant may be present in a maximum concentration of 30% by weight, such as a concentration ranging from 0.5% tol 0% by weight, relative to the total weight of the composition.

The basic compositions may also comprise at least one treating agent of cationic, anionic, nonionic or amphoteric nature to improve the cosmetic properties of the hair or to attenuate or avoid its degradation.

Suitable treating agents include, but are not limited to, those described in French Patent Nos. 2,598,613 and 2,470,596. It is also possible to use as treating agents volatile or non-volatile silicones, linear or cyclic silicones and mixtures thereof, polydimethylsiloxanes, quaternized polyorganosiloxanes such as those described in French Patent Application No. 2,535,730, polyorganosiloxanes containing aminoalkyl groups modified with alkoxycarbonylalkyl groups, such as those described in U.S. Pat. No. 4,749,732, polyorganosiloxanes such as the polyoxyalkyl polydimethylsiloxane copolymer of the dimethicone copolyol type, a polydimethylsiloxane containing stearoxy end groups (stearoxy dimethicone), a dialkylammonium acetate polydimethylsiloxane or a polydimethylsiloxane polyalkylbetaine copolymer described in British Patent No. 2,197,352, polysiloxanes organomodified with mercapto or mercaptoalkyl groups, such as those described in French Patent No. 1,530,369 and in European Patent Application EP 295,780, and also silanes such as stearoxytrimethylsilane.

The basic compositions may also comprise other treating ingredients such as cationic polymers, for instance those used in the compositions of French Patent Nos. 79/32078 (2,472,382) and 80/26421 (2,495,931); ionene cationic polymers, such as those used in the compositions of Luxembourgian Patent 83,703; basic amino acids, such as lysine or arginine; acidic amino acids, such as glutamic acid or aspartic acid; peptides and derivatives thereof; protein hydrolysates; waxes; swelling agents, penetrating agents or agents that reinforce the efficacy of the reducing agent, such as a SiO₂/PDMS (polydimethylsiloxane) mixture, dimethylisosorbitol, urea and its derivatives, pyrrolidone, N-alkylpyrrolidones, thiamorpholinone, alkylene glycol or dialkylene glycol alkyl ethers, for instance propylene glycol monomethyl ether, dipropylene glycol monomethyl ether, ethylene glycol monoethyl ether and diethylene glycol monoethyl ether, C3-C6 alkanediols, for instance propane-1,2-diol, propane-1,3-diol and butane-1,2-diol, and 2-imidazolidinone; other compounds such as fatty alcohols and lanolin derivatives; active ingredients such as pantothenic acid; agents for preventing hair loss; antidandruff agents; thickeners; suspending agents; sequestering or complexing agents; opacifiers; sunscreens; fragrances; and preserving agents.

In some embodiments, the compositions are in aqueous form, for example, in the form of a thickened or unthickened lotion, a cream or a gel.

In some embodiments, the compositions may also comprise solvents, such as ethanol, propanol, isopropanol, butanol, and glycerol, at a maximum concentration of 20% by weight, relative to the total weight of the composition.

The cosmetically acceptable medium of the compositions may be chosen from water and aqueous-alcoholic solutions of a C₂-C₈ alcohol, such as ethanol and isopropanol.

The compositions may also be in the form of thickened creams so as to hold the hair in the final desired shape. These creams are made in the form of “heavy” emulsions, for example, based on glyceryl stearate, glycol stearate, self-emulsifying waxes, or fatty alcohols.

Liquids or gels containing thickeners, such as carboxyvinyl polymers or copolymers that “stick” the hairs together and hold them in the desired shape during the leave-in time, may also be used.

The compositions may also comprise at least one adjuvant chosen from silicones in soluble, dispersed or microdispersed form; nonionic, anionic, cationic and amphoteric surfactants; ceramides, glycoceramides and pseudoceramides; vitamins and provitamins including panthenol; plant, animal, mineral and synthetic oils; waxes other than ceramides, glycoceramides and pseudoceramides; water-soluble and liposoluble, silicone-based or non-silicone-based sunscreens; nacreous agents and opacifiers; sequestering agents; plasticizers; solubilizers; acidifying agents; mineral and organic thickeners; antioxidants; hydroxy acids; penetrating agents; fragrances; and preserving agents.

The present disclosure also relates to kits comprising at least two compartments, one of the compartments (i) comprising, in a cosmetically acceptable medium, at least one imine not belonging to the hydroxide family, the cosmetically acceptable medium and the at least one imine being chosen such that the at least one imine is reactive with the cystines of keratin fibers, via a beta-elimination reaction, to produce dehydroalanine and lead to the formation of lanthionine, to produce with the keratin fibers curls ranging from 0.2 cm and 3 cm in diameter in less than 60 minutes.

The kits may also comprise an additional composition (ii) for caring for, conditioning, making up, removing makeup from, protecting, cleansing (washing) keratin fibers.

The compositions of the kits may be packaged in separate compartments, containers or devices, optionally accompanied by suitable, identical or different application means, such as fine brushes, coarse brushes, and sponges.

The disclosure also provides processes for permanently shaping keratin fibers using a cosmetic composition comprising, in a cosmetically acceptable medium, at least one imine not belonging to the hydroxide family, wherein the cosmetically acceptable medium and the at least one imine are chosen such that the imine reacts with the cystines of the keratin fibers, via a beta-elimination reaction to produce dehydroalanine and lead to the formation of lanthionine, to produce with the keratin fibers curls ranging from 0.2 cm to 3 cm in diameter in less than 60 minutes.

In some embodiments, the permanent shaping time is less than 40 minutes, or even less than 30 minutes.

In the permanent shaping processes described herein, the composition is applied to wet or dry hair, wound beforehand onto rollers (curlers) having a diameter of from 2 mm to 30 mm. It is also possible to apply the composition as the hair is wound up. After the composition has been left to act for a period ranging from 5 to 60 minutes, for example from 5 to 40 minutes, the rollers are removed, and the hair is rinsed abundantly.

After applying the composition described herein, the keratin fibers may be subjected to a heat treatment by heating to a temperature ranging from 30° C. to 60° C. In practice, this operation may be performed using a hairstyling hood, a hairdryer, an infrared ray dispenser, and other standard heating devices.

It is also possible to use, as a means of both heating and shaping the hair, a hot iron at a temperature ranging from 60° C. to 220° C., for example from 120° C. to 200° C.

The disclosure also relates to the use of an imine not belonging to the hydroxide family as an active agent for permanently shaping keratin fibers.

The disclosure also relates to an active agent for permanently shaping keratin fibers, by means of a beta-elimination reaction producing dehydroalanine and leading to the formation of lanthionine, comprising at least one imine not belonging to the hydroxide family.

The disclosure may be understood more clearly with the aid of the non-limiting examples that follow. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE 1

A simplified permanent-waving composition was prepared, containing, as a permanent-waving active agent, 1,1-dimethylbiguanide (Registry Number [1115-704]) at a concentration of 0.7M in water. The pH of the composition was 13.6. This composition was applied to Caucasian hair, wound beforehand onto curlers, for 15 minutes at a temperature of 30° C. The hair was beautifully curly, and felt soft.

### EXAMPLE 2

A simplified permanent-waving composition was prepared, containing, as a permanent-waving active agent, 1,1-dimethylbiguanide (Registry Number [1115-70-4]) at a concentration of 0.5M in water. The pH of the composition was 13.5. This composition was applied to Caucasian hair, wound beforehand onto curlers, for 25 minutes at a temperature of 30° C. The hair was beautifully curly and felt soft.

### EXAMPLE 3

A simplified permanent-waving composition was prepared, containing, as a permanent-waving active agent, 1,3,4,6,7,8-hexahydro-2H-pyrimido[1,2-a]pyrimidine (Registry Number [5807-14-7]) at a concentration of 0.7M in water. The pH of the composition was 13.5. This composition was applied to Caucasian hair, wound beforehand onto curlers, for 15 minutes at a temperature of 30° C. The hair was beautifully curly and felt soft.

### EXAMPLE 4

A simplified permanent-waving composition was prepared, containing, as a permanent-waving active agent, 1,3,4,6,7,8-hexahydro-2H-pyrimido[1,2-a]pyrimidine (Registry Number [5807-14-7]) at a concentration of 0.5M in water. The pH of the composition was 13.4. This composition was applied to Caucasian hair, wound beforehand onto curlers, for 20 minutes at a temperature of 30° C. The hair was beautifully curly and felt soft.

1. A cosmetic composition comprising, in a cosmetically acceptable medium, at least one imine not belonging to the hydroxide family, the cosmetically acceptable medium and the at least one imine being chosen such that the at least one imine is reactive with the cystines of keratin fibers via a beta-elimination reaction to produce dehydroalanine and lead to formation of lanthionine, to produce keratin fiber curls ranging from 0.2 cm to 3 cm in diameter in less than 60 minutes.

2. The composition according to claim 1, wherein the curls are produced in less than 40 minutes.

3. The composition according to claim 2, wherein the curls are produced in less than 30 minutes.

4. The composition according to claim 1, wherein the at least one imine is present in a concentration ranging from 0.01 M to 4M.

5. The composition according to claim 4, wherein the at least one imine is present in a concentration ranging from 0.05M to 2M.

6. The composition according to claim 1, wherein the pH ranges from 9.6 to 14.

7. The composition according to claim 6, wherein the pH ranges from 11 to 13.

8. The composition according to claim 1, wherein the composition comprises 0% of base belonging to the hydroxide family.

9. The composition according to claim 1, further comprising at least one adjuvant chosen from silicones in soluble, dispersed or microdispersed form; nonionic, anionic, cationic and amphoteric surfactants; ceramides; glycoceramides; pseudoceramides; vitamins and provitamins; plant, animal, mineral and synthetic oils; waxes other than ceramides, glycoceramides and pseudoceramides; water-soluble and liposoluble, silicone-based or non-silicone-based sunscreens; nacreous agents; opacifiers; sequestering agents; plasticizers; solubilizers; acidifying agents; mineral and organic thickeners; antioxidants; hydroxy acids; penetrating agents; fragrances; and preserving agents.

10. The composition according to claim 1, wherein the at least one imine is chosen from guanidine and amidine imines.

11. The composition according to claim 10, wherein the guanidine imines are chosen from:
N,N-dimethylguanidine,
N,N′,N″-trimethylguanidine,
N-methylguanidine,
N,N-diethylguanidine,
N-ethylguanidine,
N,N-dimethylimidodicarbonimidic diamide,
N-(tert-butyl)-N″-methylguanidine,
N-(4-aminobutyl)guanidine,
1,3,4,6,7,8-hexahydro-2H-pyrimido[1,2-a]pyrimidine,
N,N″-diisopropylguanidine,
1-methyl-1,3,4,6,7,8-hexahydro-2H-pyrimido[1,2-a]pyrimidine,
N″-(tert-butyl)-N,N,N′,N′-tetramethylguanidine,
N,N,N′,N′-tetraethylguanidine,
N,N″-di-tert-butylguanidine,
N″-[amino(imino)methyl]-N-phenylguanidine,
N, N′,N″-triisopropylguanidine,
pyrrolidine-1-carboximidamide,
N-phenylguanidine,
N-(tert-butyl)-N′,N″-dimethylguanidine,
N-(4-{[amino(imino)methyl]amino}butyl)guanidine,
N″-(tert-butyl)-N-methyl-N′-phenylguanidine,
N,N′,N″-tricyclohexylguanidine,
N-{3-[(4-{[amino(imino)methyl]amino}butyl)amino]propyl}guanidine,
N-(2-hydroxyethyl)-N-methylguanidine,
n-propylguanidine,
isopropylguanidine,
(2-methylpropyl)guanidine,
(2-ethylhexyl)guanidine,
(2-hydroxyethyl)guanidine,
(2-methoxyethyl)guanidine,
o-, m- or p-hydroxybenzylguanidine,
cyclopentylguanidine,
cyclohexylguanidine,
cyclohexylmethylguanidine,
4-oxacyclohexylguanidine,
glucopyranosylguanidine,
biguanido(guanylguanidine),
1-methylbiguanide,
1-ethylbiguanide,
1-n-propylbiguanide,
1-isopropylbiguanide,
1-(2-methylpropyl)biguanide,
1-(2-hydroxyethyl)biguanide,
1-phenylbiguanide,
1-benzylbiguanide,
1-cyclopentylbiguanide,
1-cyclohexylbiguanide,
1,1-dimethylbiguanide,
1,1-diethylbiguanide,
1-ethyl-1-methylbiguanide,
1-methyl-1-ethylguanidine,
1-methyl-1-n-propylguanidine,
1-methyl-1-isopropylguanidine,
1,1-bis(2-hydroxyethyl)guanidine,
1-methyl-1-phenylguanidine,
3-methyl-1-ethylguanidine,
3-methyl-1-n-propylguanidine,
3-methyl-1-isopropylguanidine,
1,3-bis(2-hydroxyethyl)guanidine,
1,3-dibenzylguanidine,
3-methyl-1-benzylguanidine,
1,3-dicyclopentylguanidine,
3-methyl-1-cyclopentylguanidine,
3-methyl-1-cyclohexylguanidine,
1,3-di(4-oxacyclohexyl)guanidine,
3-methyl-1-(4-oxacyclohexyl)guanidine,
3-methyl-1-(glucopyranosyl)guanidine, and
N-[bis(dimethylamino)methylene]-N′,N″-diisopropylguanidine.

12. The composition according to claim 10, wherein the amidine imines are chosen from:
1,2-dimethyl-1,4,5,6-tetrahydropyrimidine,
1-methyl-2-(2-thiophen-2-ylvinyl)-1,4,5,6-tetrahydropyrimidine,
2,3,4,6,7,8,9,10-octahydropyrimido[1,2-a]azepine,
2,3,4,6,7,8-hexahydropyrrolo[1,2-a]pyrimidine,
2-methylpropanimidamide,
2-methylpropionamidine,
3-amidinopyridine,
3-pyridinecarboxamidine,
6-(dibutylamino)-1,8-diazobicyclo[5.4.0]undec-7-ene,
acetamidine,
butyramidine, and
propionamidine.

13. A kit comprising at least two compartments, wherein one of the compartments comprises a first composition comprising, in a cosmetically acceptable medium, at least one imine not belonging to the hydroxide family, the cosmetically acceptable medium and the at least one imine being chosen such that the at least one imine is reactive with the cystines of keratin fibers, via a beta-elimination reaction to produce dehydroalanine and lead to formation of lanthionine, to produce keratin fiber curls ranging from 0.2 cm to 3 cm in diameter in less than 60 minutes.

14. The kit according to claim 13, further comprising a second composition for caring for, conditioning, making up, removing makeup from, protecting, cleansing, or washing keratin fibers.

15. A process for permanently shaping keratin fibers comprising applying to the keratin fibers a cosmetic composition comprising, in a cosmetically acceptable medium, at least one imine not belonging to the hydroxide family, wherein the cosmetically acceptable medium and the at least one imine are chosen such that the at least one imine reacts with the cystines of the keratin fibers, via a beta-elimination reaction to produce dehydroalanine and lead to formation of lanthionine, to produce keratin fiber curls ranging from 0.2 cm to 3 cm in diameter in less than 60 minutes.

16. The process for permanently shaping keratin fibers according to claim 15, further comprising subjecting the keratin fibers to a heat treatment by heating the keratin fibers to a temperature ranging from 36° C. to 60° C.

17. The process for permanently shaping keratin fibers according to claim 16, wherein the heat treatment comprises heating the keratin fibers with a hot iron at a temperature ranging from 60° C. to 220° C.

18. The process for permanently shaping keratin fibers according to claim 17, wherein the iron temperature ranges from 120° C. to 200° C.

19. The process for permanently shaping keratin fibers according to claim 15, wherein the keratin fibers are permanently shaped in less than 40 minutes.

20. The process for permanently shaping keratin fibers according to claim 19, wherein the keratin fibers are permanently shaped in less than 30 minutes.

21. An active agent for permanently shaping keratin fibers, wherein said active agent comprises at least one imine not belonging to the hydroxide family, wherein the at least one imine is reactive with the cystines of the keratin fibers, via a beta-elimination reaction to produce dehydroalanine and lead to formation of lanthionine, to permanently shape the keratin fibers.
